(19) United States
(12) Patent Application Publication
(10) Pub. No.: US 2024/0138477 A1
(43) Pub. Date: May 2, 2024

(54) DEVICE FOR VOLATILISATION OF AN AQUEOUS SOLUTION AND METHOD THEREOF

(71) Applicant: Avogen Tech SA, Monthey (CH)

(72) Inventors: Yannick Gendre, Fully (CH); David Avolio, Collombey (CH)

(73) Assignee: Avogen Tech SA, Monthey (CH)

(21) Appl. No.: 18/281,256

(22) PCT Filed: Mar. 2, 2022
(86) PCT No.: PCT/IB2022/051822

§ 371 Date: Sep. 8, 2023

(30) Foreign Application Priority Data
Mar. 9, 2021 (EP) 21161408.6

(51) Int. Cl. A24F 40/46; A24B 15/167; A24F 40/05; A24F 40/10; A24F 40/60

(52) U.S. Cl. CPC A24F 40/46; A24B 15/167; A24F 40/05; A24F 40/10; A24F 40/60

(57) ABSTRACT

The present invention relates to a device (1) adapted to volatilize a viscous liquid composition (S) into microdroplets (5) comprising a pre-heating chamber (12) upstream a volatilizing element. It further relates to a method of providing an aerosol from a liquid composition, aqueous liquid compositions used for the production of aerosols, and to a kit comprising the same.

### TECHNICAL FIELD

The present invention concerns a vaporizing device such as an electronic cigarette or an inhalator. In particular, the device according to the present invention is adapted for the vaporization of aqueous solutions with an improved efficiency. It allows to produce microdroplets having a mean diameter lower than around 4 μm, preferably in the order of 1 μm. The present disclosure further concerns a process for vaporizing an aqueous solution in an aerosol of such droplets. It also concerns a kit comprising the device and at least one aqueous solution separately packed.

### STATE OF THE ART

It is known that the mean size of the droplets is an important parameter for the efficiency of an inhaled substance. Microdroplets of around 4 μm are usually considered optimal to ingress through the lung. Whether the substance, or the solution wherein it is solved, relates to a pharmaceutical product or to a tobacco derivative such as nicotine, it is preferable to produce an aerosol having homogenous particle size of a diameter lower than around 4 μm.

The substances used for inhalation are often combined with additives or organic solvents for stability or homogeneity reasons. For the purpose of vaporization, such non-aqueous solvents are usually more favorable for producing low size microdroplets due to their low natural viscosity. However, the additives or organic solvents may provide some negative effects. Beside the potential non desirable interaction with the organism, such non aqueous solutions can provide irritations, bad taste, or any other detrimental sensations once inhaled or during inhalation. Aqueous solutions thus appear preferable for the inhalation. Due to the natural Lower viscosity of the water compared to organic solvent, it is difficult to produce an aerosol having the requested particle mean size of lower than 4 μm.

The document WO9501137 describes a device allowing to heat the substance in solution until it vaporizes. Such a method does not appear suitable for aqueous solutions because the high enthalpy of vaporization of the water requests to delivering locally a high amount of energy to vaporize the solvent. This results in a high consumption of energy and of a long laps time before vaporization occurs. In addition, temperature may degrade the substances of the composition, or induce non desirable chemical reaction in the mixture.

The document US2019014819 describes a device allowing to volatilize aerosol precursors by means of a piezoelectric mesh so as to avoid any side effects resulting from a heating process. Although compositions based on organic solvents can be volatilized, such arrangement does not appear directly applicable for aqueous solutions. In particular, it is non optimal for controlling the mean size of the droplets of an aqueous composition. In addition, the piezoelectric mesh necessitates an electrical boost to vaporize the solution, which represent a limitation in term of autonomy, when the device is fed by a battery.

### BRIEF SUMMARY OF THE INVENTION

It is thus an object of the present invention to provide a device allowing to nebulize an aqueous solution with a controlled droplet size. In particular, it is an aim of the present invention to provide a device allowing to nebulize aqueous solutions in an aerosol of droplets having a mean diameter lower than 4 μm, or in the order of 1 μm or lower.

The aim is also to provide a device allowing to reduce the viscosity of liquid compositions below 1 cP so that the flow rate of the resulting aerosol is improved.

Another aim of the present invention is to provide a device allowing to volatilize an aqueous solution without burning or degrading the substances it comprises. It is thus an object to volatilize the aqueous solution without boiling the solution.

Another object of the present disclosure is to provide a process for vaporizing an aqueous solution into an aerosol of droplets having a controlled means size. It is more particularly an object of vaporizing the aqueous solution while controlling the mean size of the particles.

Another object of the present invention is to provide a device adapted for volatilizing an aqueous solution and allowing to modulated the temperature, the size of the emitted droplets or the duration of the volatilization.

It is a further object to provide an aqueous solution comprising at least one substance having biological effects, the aqueous solution having a viscosity higher than around 2, or 1.5 or 1.4 cP at 20° C. at room temperature. The aim is more particularly to provide a solution comprising less organic solvent as known solution, or free of, or substantially free of organic solvents. The aim is to provide a liquid solution having less that 30%, or less than 25% or 20% of organic solvent, for inhalation purpose.

These objectives are realized by the means of the invention described in the independent claims, and further detailed in the dependent claims.

The claimed solution solves the above-mentioned drawbacks. In particular, it allows to spread microdroplets of aqueous solutions with a controlled average diameter. It furthermore improves the efficiency of the produced aerosol, which are free or substantially free of undesirable additive or solvent. It further allows to volatilize substances with a minimum energy consumption. The claimed device has thus a longer autonomy and presents an improved comfort of use. The claimed solution allows also to control user taste parameters via the flavour enhancer assay and heat via the temperature of the post heater.

### BRIEF DESCRIPTION OF THE FIGURES

Example of realization of the presently described invention are illustrated by the following drawings:

FIG. 1: schematic view of the device according to the present invention.

FIG. 2: details of the pre-heating chamber of the device according to the present invention.

FIG. 3: Schematic command diagram for a device according to the present invention.

FIG. 4a: Section view of the device of the present description according to an embodiment.

FIG. 4b: Section view of the device of the present description according to an embodiment.

FIG. 5a: Section view of the device of the present description according to an embodiment.

FIG. 5b: Section view of the device of the present description according to an embodiment.

### EMBODIMENTS OF THE INVENTION

The device 1 of the present disclosure comprises a housing 10 having a storage compartment 11. The storage compartment 11 may be an internal space of the housing 10, for example an internal space at an extremity of the housing 10. To access the storage compartment 11, the housing 10 comprises a removable part such as a cap or a lid which can be screwed or clipped at the extremity of the housing 10. Alternatively, a portion of the housing 10 can be separated from the rest of the device to open the access to the storage compartment 11. Such a removable moiety may represent a portion of the device such as 10% or 20% of its length. It can be for example screwed on the remaining moiety of the housing 10, representing the body of the device 1. Alternatively, the storage compartment 11 may be a central compartment of the device which can be accessed by means of an openable lateral lid. Such a lateral lid can be linked to the housing 10 through one or several hinges.

The storage compartment 11 may be adapted to receive a liquid composition S from an external tank. Suitable sealings are arranged at the connection between the lid and the housing 10 so as to avoid leakage. According to an embodiment, the housing 10 comprises at least a transparent portion, such as a lateral window, at the level of the storage compartment 11 to allow the user visualizing the level of liquid composition S remaining in the storage compartment 11. The housing portion corresponding to the storage compartment 11 may thus be fully transparent or transparent on a portion of its surface, such as on half of its surface. Alternatively, only the lid covering the storage compartment 11 is transparent.

According to another embodiment, the storage compartment 11 may be adapted to receive a cartridge of a liquid composition S. Such a cartridge has an internal volume adapted to store a liquid formulation and a connection part adapted to couple the cartridge to the device 1. The connection part may comprise a sealing means preventing the leakage of the liquid formulation S when the cartridge is separated from the device 1. It can in addition comprise a fixation means such as a thread or one or several lugs adapted to maintain the cartridge connected to the device 1. The sealing means of the cartridge is preferably adapted to automatically open once the cartridge is connected to the device 1. To this end. A channel extremity may be present on the device 1, at the connection point of the cartridge, which can proceed to perforation or opening of the sealing means of the cartridge when coupling it to the device 1. In case the storage compartment 11 is adapted for receiving a cartridge, it may be free of lid or cap, so that the cartridge can be directly connected to the housing 10 without too many manipulations. Alternatively, a cap may be provided at least to protect the connection part of the device 1. If present, a cap may also serve at exerting the necessary force to the cartridge for perforation or opening of the sealing means upon coupling to the device 1. It is here understood that any suitable arrangement can be adapted to the present device 1.

The storage compartment 11 comprises at least one solution inlet 110 allowing the liquid composition S flowing from the storage compartment 11 to a pre-heating chamber 12. The solution inlet 110 may be a through hole or a plurality of through holes allowing the liquid composition S flowing freely to the pre-heating chamber 12. Such an arrangement may however be sensitive to the gravity and limit the number of positions the device 1 can take under using conditions. The solution inlet 110 may comprise a movable part allowing to close and open it on demand. For example, the pre-heating chamber 12 may have a predetermined volume corresponding to a dose of solution S to be inhaled. By opening the solution inlet 110, the pre-heating chamber 12 can be filled with a suitable volume of liquid solution S. Closing the solution inlet 110 before initiating the inhalation then guaranties that no excess of composition S is inhaled.

Alternatively, the solution inlet 110 can comprise one or several pores of limited size through which the liquid composition S can flow by capillarity. The size and the number of the pores may be adapted to the viscosity of the liquid composition S. According to a preferred arrangement, the diameter of the pores is calibrated to prevent or substantially prevent the passage of a liquid having a viscosity higher than around 3 cP or 5 or 10 cP. Alternatively or in addition, the size of the pore is calibrated to allow or substantially allow the progression of a liquid composition by capillarity having a viscosity lower than around 2 cP, or lower than around 1.5 cP, or even lower than 1 cP. According to preferred arrangement, the size of the pores may be calibrated so that the progression by capillarity of a liquid composition having a viscosity lower than 1 cP is 2 or 3 or 4 time faster than the progression of a liquid composition having a viscosity of more than 3 or 4 cP. Alternatively, one or more of the pores of the solution inlet 110 may be larger to allow the free passage of the liquid composition S, independently of its viscosity, and comprise a cotton wick adapted to absorb the liquid composition S by capillarity.

The device 1 of the present disclosure comprises a pre-heating chamber 12 downstream the storage compartment 11. The pre-heating chamber 12 comprises or is in direct contact with at least one heating device 120. Such a heating device may be an electrical resistance, or any related device adapted to deliver some heat. The heating device 120 may be placed inside the pre-heating chamber 12 so that it is in direct contact with the liquid composition S. It thus allows a very efficient and rapid local heating of the liquid composition 1 surrounding the heating device 120. The heating device 120 may be positioned closer the solution inlet 110 so that the liquid composition S is already warmed when flowing to the pre-heating chamber 12. Alternatively, the heating device 120 may be placed downstream, closer a volatilizing element so that the liquid composition S is warmed locally just before being spread as microdroplets 5 by the volatilizing element.

The heating device 120 may be encapsulated into a protective housing, which is immerged in the liquid composition S inside the pre-heating chamber 12. Such a protective housing may be of thermo-conductive material such as a metal, like stainless steel, aluminum or similar metallic element. The protective housing may in addition comprise extensions allowing to homogenize the temperature through the volume of the pre-heating chamber 12. Alternatively, the protective housing may be in clause contact with one or several walls of the pre-heating chamber 12.

Alternatively, the heating device 120 may be integrated within one or several walls of the pre-heating chamber 12 such as the lateral walls, or the wall separating the storage compartment 11 from the pre-heating chamber 12, or the wall separating the pre-heating chamber 12 from an aerosol chamber 13.

According to an embodiment, the heating device 120 is integrated within the solution inlet 110 so that the liquid composition S enters in the pre-heating chamber at a suitable temperature.

According to another embodiment, the heating device 120 is integrated to the downstream wall of the pre-heating chamber 12 so that the heating energy is provided at or close to the volatilizing point of the liquid composition S.

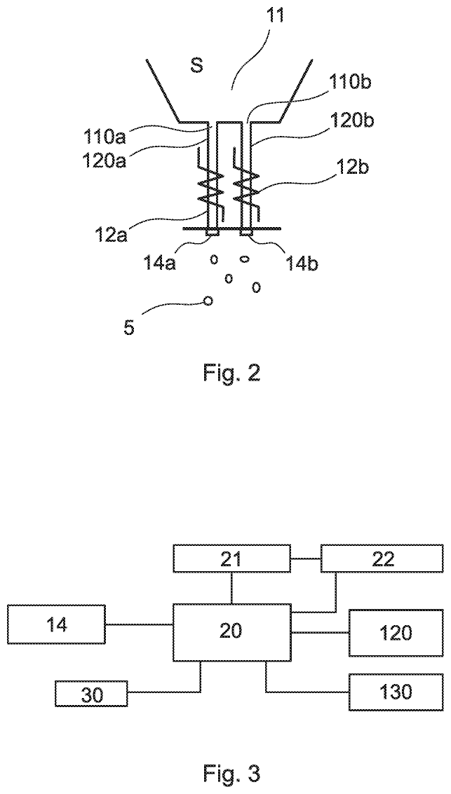

According to an embodiment better shown on FIG. 2, the pre-heating chamber 12 comprises a plurality of capillaries 12a, 12b fluidly connected to the solution inlet 110 at one of their extremity and fluidly connected to the volatilizing element at their opposite extremity. The internal diameter of the capillaries 12a, 12b may be of few hundreds of micrometers. It may be comprised for example between around 100 micrometers and around 800 micrometers, or around 200 micrometers and around 600 micrometers. The internal diameter of the capillaries 12a, 12b may decrease along their length from the solution inlet 110 to the volatilizing element. This allows to control the progression of the liquid composition S along the capillaries according to its viscosity. The internal diameter of the capillaries 12a, 12b may then decrease by a value comprised between 10% and 50%, such as around 20% or 30%, from one extremity to the other one. One or several heating devices 120 may surround the capillaries 12a, 12b. The heating devices 120 can be in direct contact with the capillaries for a fast and efficient temperature control. The heating devices 120 can alternatively be distant from the walls of the capillaries, allowing a smooth temperature variation preventing local burning of the components of the liquid composition S or preventing local boiling or pressure increase of the liquid composition S. The combination of the heating device 120 and the capillaries having either constant internal diameter or decreasing internal diameters, allows to control the progression speed of the liquid composition S, thus influencing its debit.

The heating device 120 is adapted to heat the liquid composition S at a temperature which prevents its boiling, the burning of its components, side reactions or chemical degradation thereof. The liquid composition S is thus preferably heated at a temperature comprised between around 30° C. and around 90° C., preferably lower than 80° C. or 60° C. A temperature comprised between around 40° C. and 60° C. appears suitable for most of the liquid compositions S that are envisaged for the present device 1. According to another aspect, the heating device 120 is controlled to give the liquid composition S the suitable viscosity. The viscosity of the liquid solutions S, in particular aqueous solutions, is usually too high at 20° C. to provide droplet sufficiently small and/or a suitable debit. Also, the external usage of the device 1, in particular during wintertime, may occur at very low temperatures, which naturally significantly increase the viscosity of the liquid compositions. The heating device 120 thus help to regulate and reproduce the suitable viscosity with a minimum of energy consumption.

It is highlighted that capillaries 12a, 12b allow fast temperature exchanges, providing an immediate control of the debit and/or temperature of the liquid solution in the pre-heating chamber 12. It is also highlighted that the length of the capillaries 12a, 12b across the pre-heating chamber 12 also influences the temperature control of the liquid composition S. The capillaries 12a, 12b may have a straight shape from the solution inlet 110 to the volatilizing element. For a better temperature control, they can alternatively exhibit a longer pathway in the pre-heating chamber 12 by means of a non-straight shape. The capillaries 12a, 12b can for example have the shape of a coil or comprise baffles or take any suitable serpentine pathway allowing an efficient temperature exchange with the heating device 12.

According to an embodiment, the internal volume of the pre-heating chamber 12 comprises a porous material allowing to contain and drain the liquid composition S. the heating device 12, may be arranged as above-mentioned, or inside the porous material comprising the liquid composition S. The porous material can thus be maintained at the suitable temperature. Such porous material may be in direct contact with the volatilizing element.

The device 1 according to the present disclosure further comprises a volatilizing element adapted to spread microdroplet 5 toward the aerosol chamber 13 downstream the pre-heating chamber 12 from the liquid composition S present in this pre-heating chamber 12. The volatilizing element comprises at least one mesh 14 through which the liquid composition S can pass while forming microdroplets 5. The pores of the mesh 14 has a diameter preferably comprised between 0.5 micrometers and few micrometers like 3 to 10 micrometers. The mesh 14 may extend through all the surface between the pre-heating chamber 12 and the aerosol chamber 13, thus presenting a homogenous porosity on the entire surface. Such arrangement appears convenient when the surface between the pre-heating chamber 12 and the aerosol chamber 13 is completely or substantially completely in contact with the liquid composition S. It is such a case when the volume of the pre-heating chamber 12 is filled with the liquid composition S or when a porous material is in contact with the mesh 14.

Alternatively, several meshes 14a, 14b can be dispatched on the surface separating the pre-heating chamber 12 and the aerosol chamber 13, forming the only passages allowing the liquid composition S passing toward the aerosol chamber 13. Such a configuration is advantageous in combination with capillaries above described 12a, 12b. The extremity of the capillaries 12a, 12b coincides with the corresponding meshes 14. The liquid composition S, continuously flowing through the capillaries arrives in direct contact with the meshes 14 and can be spread as microdroplets 5 into the aerosol chamber 13. The number and size of such meshes depends on the needs.

Whether the mesh 14 is a unique mesh covering the surface between the pre-heating chamber 12 and the aerosol chamber 13, or whether it corresponds to a plurality of small meshes, it may be integrated or combined to a vibrating actuator. For example, the mesh 14 can be integral with a flexible membrane combined to a vibrating actuator. The vibration frequency provided by the vibrating actuator may be comprised between around 50 and 150 kHz. The vibration frequency provided by the vibrating actuator is preferably comprised 80 and 130 KHz, more preferably between 100 and 120 kHz.

The number of holes of the mesh 14, or of the plurality of meshes is comprised between 500 and 5000, preferably between 800 and 2000. The number of holes spread on the surface between the pre-heating chamber 12 and the aerosol chamber 13 is for example around 1000 holes.

Alternatively, an outlet comprising a vibrating actuator may be arranged upstream the mesh 14 so as to actively direct the liquid composition S from the pre-heating chamber toward the aerosol chamber 13 through the mesh 14. Such a vibrating outlet relates to a pump. The vibration frequency is in this configuration higher than 500 KHz. The frequency may be for example comprised between around 500 and 1500 KHz or between around 500 and 1000 KHz.

In addition, a mesh 14 may comprise several layers of porous material, either combined together or separated by an interstitial space. The layers of porous material may exhibit different pore size. The pore size of the first layer, placed upstream the liquid pathway can then be larger than the pore size of the last layer, placed downstream. The liquid fragmentation can then be progressive along the pathway, allowing to reduce the applied energy necessary for volatilizing the liquid composition. The vibration frequency can be to this end lowered.

A mesh 14 according to the present disclosure may alternatively or in addition comprise non-flat portions of porous material, so that the contact surface with the liquid composition S is increased. The mesh 14 may have a tridimensional shape. It can comprise for example folded sheets.

Pipes, preheating chamber, vibrating mesh chamber and a mesh according to the present disclosure may alternatively or in addition have a smooth surface limiting or avoiding the deposit of residues. It may in addition be coated with a hydrophobic material or an antiadhesive material or any other material limiting the adhesion of residues. Alternatively, the surfaces can be coated with silver, which has in situ bactericide properties.

The vibrating actuator may be any device allowing the vibration of the mesh 14 at the suitable frequencies. It can be for example a piezoelectric or a piezomagnetic actuator or related device. It can in addition comprise several vibrating actuators either identical or different, arranged in parallel.

Downstream the volatilizing element is an aerosol chamber 13 wherein the microdroplets 5 emanating from the volatilizing element are spread. The mean size of the microdroplets 5 is preferably below 4 micrometers, even more preferably below 2 micrometers. It can be around 1 micrometer for a good inhalation. It is indeed an objective of the present device to provide thin microdroplets which can easily flow as far as possible into the lung.

The aerosol chamber 13 can comprise an air inlet 6 allowing to intake external fresh air while inhaling the liquid composition S. The air inlet 6 may comprise a valve allowing to open and close it. Such a valve may be tuned to adapt the ratio of fresh air intake compared to the internal aerosol of microdroplets 5. The ratio of fresh air to microdroplets 5 can be for example around 50/50. Alternatively the inhaled volume comprises more fresh air than microdroplets with a ratio of fresh air to microdroplets 5 higher than 60/40 or even higher 80/20. Alternatively, the inhaled volume comprises less fresh air than microdroplets with a ratio of fresh air to microdroplets 5 lower than 40/60 or even lower than 20/80.

The air inlet 6 may also comprise a filter to avoid inhalation of impurities and microparticles suspended in the air. The aerosol of microdroplet 5 is thus mixt with external air in a predetermine proportion when the user inhales the liquid composition S.

The air inlet 6 may be visible on the housing 10 of the device 1 at the level of the aerosol chamber 13. Alternatively, the air inlet 6 is visible on the housing 10 at the level of the volatilizing element, or at the level of the pre-heating chamber 12, or at the level of the storage compartment 11. This means that the external air travels inside the housing 10 of the device 1 from the air inlet 6 to the aerosol chamber 13. It can for example travel in a double wall around the pre-heating chamber 12. The intake air can then be pre-heated as well as the liquid composition S, due to its proximity with the pre-heating chamber 12.

The aerosol chamber 13 also comprises a mouth port 9 allowing the user inhaling the liquid composition S.

The aerosol chamber 13 may in addition comprise one or several aerosol heater 130. Any device providing heat, such as an electrical resistance, can be used as such. The aerosol heater 130 may be placed close to the air inlet 6 so as to pre-heat the external air at a suitable temperature, in particular to avoid condensing the microdroplets 5. Such an arrangement is particularly suitable when the air inlet 6 is directly placed on the aerosol chamber 13, since it does not flow around the pre-heating chamber 12. In particular, the aerosol heater can be an electrical resistance, such as a metal or a coil or a metal ceramic heater, with low resistance, preferably comprised between 0.1 and around 2 ohms. the aerosol heater can be more advantageously sub ohm or a metal ceramic heater. Alternatively, the aerosol heater may be of infrared type. Such infrared type heater includes for example the polyimide or carbon infrared heaters, with wave length from around 3 to 14 μm. Infrared type heater having wave lengths corresponding to the water absorbance are preferably used.

Alternatively or in addition an aerosol heater 130 may be placed close to the mouth port 9 to guarantee that the mixture of aerosol droplets and fresh external air is inhaled at the suitable temperature.

Optionally, the aerosol chamber 13 can comprise a means to collect the condensed aerosol which has not been inhaled by the user. The size of the droplets of the aerosols is designed to increase or optimized the inhaled fraction versus the fraction depositing on the surfaces of the device 1. However, a small fraction of aerosol may still condensate on the internal surfaces of the aerosol chamber 13. The means adapted to collect such a condensed material may take the shape of one or several concave wall arranged at a bottom position so as to collect condensed fluids. The bottom position designates the lowest part of the device 1, either at rest or during its use or both. The means to collect the condensed fluids may further comprise one or several capillaries connected at the concave wall in a way to flow these condensed fluid back to an upstream part of the device 1. The capillaries of the collecting means can then join the concave wall of the aerosol chamber 13 to the volatilizing element. The collected condensed fluids are thus guide through the vibrating elements and volatilized again. Alternatively or in addition, the capillaries of the collecting means join the pre-heating chamber 12. The retrieved condensed fluids can thus be heated again at the proper temperature and volatilized again. Alternatively or in addition, the capillaries of the collecting means join the storage compartment 11. By this way, the retrieved condensed fluid is driven back to the full circuit.

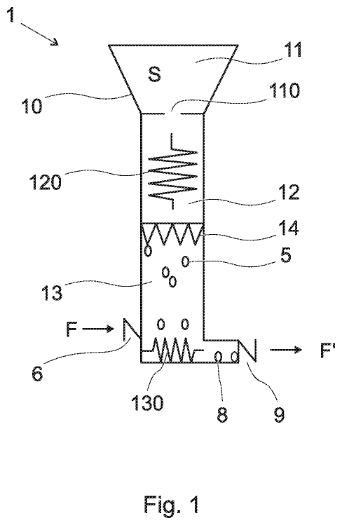

As shown in FIG. 1, the preheating chamber 12 and the aerosol chamber 13 are arranged in the continuity from one another wherein the volatilizing element, such as the vibrating mesh 14, is arranged along a transversal position crossing the space between the preheating chamber 12 and the aerosol chamber 13. Such an arrangement is well adapted for a device having the preheating chamber 12, the aerosol chamber 13 and the mouth port 9 aligned in this order along the longitudinal axis of the device 1. Such an arrangement can be for example well adapted for one shot inhalation of a product such as a drug.

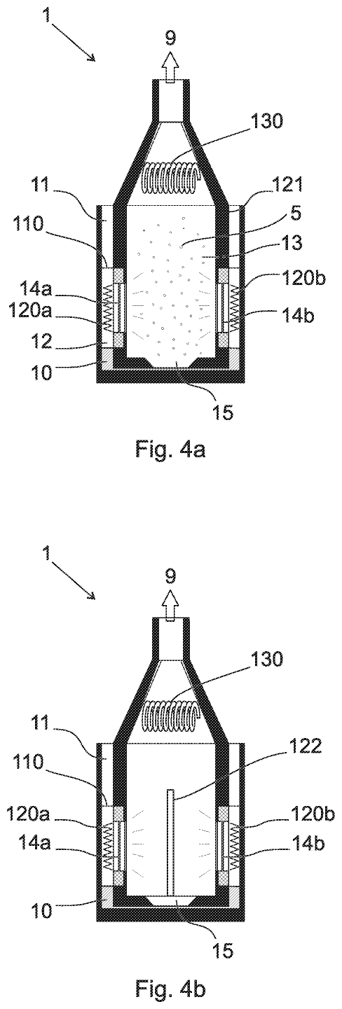

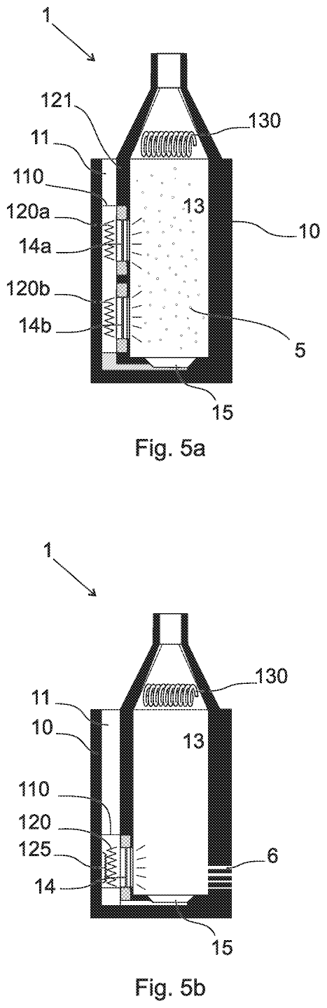

According to specific arrangements better represented in FIGS. 4a, 4b, 5a and 5b, in the device 1 according to the present disclosure, the storage compartment 11 takes the form of a lateral space between the aerosol chamber 13 and the housing 10 of the device 1. In other words, the storage compartment 11 surrounds the lateral walls 121 of the aerosol chamber 13, on a fraction of its length. For example, the storage compartment 11 can surround the lateral walls 121 of the aerosol chamber 13, on a height comprised between 10% to 80% of its length, or between 20% to 60% of its length, such as 30%, 40% or 50% of its length. It can surround the aerosol chamber 13 over all its periphery, as better shown on FIGS. 4a and 4b, or only on a portion of its periphery, as better shown on FIGS. 5a and 5b.

Part of the aerosol chamber 13 is also surrounded by the preheating chamber 12, either on its entire periphery or on a portion of its periphery. The storage compartment 11 and the preheating chamber 12 are in fluid communication by means of at least one solution inlet 110. Several solution inlets 110 can be arranged around the periphery of the aerosol chamber 13, that is to say, dispatched over the common surface between the storage compartment 11 and the preheating chamber 12. The solution inlet can comprise all the features above-described. It can in particular comprise several solution inlets 110a, 110b and/or several capillaries 12a, 12b, as shown in FIG. 2. The preheating chamber 12 can extend on 10% to 80% of the length of the aerosol chamber 13 or between 20% to 60% such as around 30%, 40% or 50% of its length.

It results from such a disposition that the upper part of the lateral walls 121 of the aerosol chamber 13 is surrounded at least partly by the storage compartment 11 and the bottom part of lateral walls 121 of the aerosol chamber 13 is surrounded at least partly by the preheating chamber 12. The upper part denotes here the part which is closest from the mouth port 9 and corresponds to the orientation of the device 1 when it is used by the user. In other words, the storage compartment 11 is closer from the mouth port 9 than the preheating chamber 12, but considering the pathway of the solution S, from the storage compartment 11 to the mouth port 9, the preheating chamber 12 is still upstream the aerosol chamber 13.

One or several heating devices 120a, 120b are arranged around the lateral walls 121 of the aerosol chamber 13 in the preheating chamber 12. One or more volatilizing elements, such as the vibrating meshes 14a, 14b, are arranged in the lateral walls 121 of the aerosol chamber 13 so as to project the droplets 5 in the aerosol chamber 13. The heating devices 120a, 120b may be arranged so as to face the volatilizing elements. In that case, one or several couples of volatilizing elements and heating devices 120a, 120b surround the aerosol chamber 13. Alternatively, the heating devices 120 can be arranged within the preheating chamber 12 independently of the volatilizing elements.

Such a configuration represents the advantage of a more compact device 1, at least along its longitudinal axis. In addition, the lateral wall 121 of the aerosol chamber 13 are different and spaced from the housing 10 and are not in contact with the external environment. In particular, the storage compartment 11 and the preheating chamber 12 form a space between the housing 10 and the aerosol chamber 13. This results in a better control of the temperature inside the aerosol chamber 13 by limiting the possible temperature exchanges between the aerosol chamber and the external environment.

The volatilizing element, such as the vibrating mesh 14, is here integrated to the lateral wall 121 of the aerosol chamber 13, instead of being transversal to the lateral walls 121, as shown in FIG. 1. Although the heating device 120 is schematically represented by a resistance in FIGS. 4a, 4b, 5a and 5b, it can take any suitable form, including those described above.

Although one preheating chamber 12 is represented in FIGS. 4a, 4b, 5a and 5c, this does not exclude that several similar preheating chambers 12 are provided around the aerosol chamber 13. For example, several solution inlets 110a, 110b can lead the solution S into several preheating chambers 12 each being as described here. The corresponding vibrating meshes 14 are arranged on the lateral walls 121 and project the droplets 5 through the common aerosol chamber 13.

The lateral walls 121 of the aerosol chamber 13 can be provided with one or several volatilizing elements, such as vibrating meshes 14. Preferably, several vibrating meshes 14 are arranged on the periphery of the aerosol chamber 13.

According to a specific arrangement, the vibrating meshes 14 vibrate independently from one another. However, in particular when they are closed to each other, the combined frequency of vibration of the meshes 14 can impact the flow rate of the solution S in an uncontrolled manner. To avoid or limit the effect of an uncontrolled interaction between the vibrating meshes 14, an internal dividing wall 15 can be provided inside the preheating chamber 12 between two vibrating meshes 14 facing to each other, as better shown in FIG. 4b. Such a dividing wall 122 preferably divides only a part of the aerosol chamber 13. Although it can be plain, it can also comprises through holes so as to not completely close the space between the vibrating meshes 14. Depending on the number of vibrating meshes 14 integrated to the lateral walls 121 of the aerosol chamber 13, the dividing wall 122 can take any suitable shape. It can for example be a straight wall placed on the middle of the aerosol chamber 13 between two vibrating meshes 14 facing each other. Alternatively, it can comprise several arcuate walls each one facing a given vibrating mesh 14. Alternatively, a central cylinder can be placed inside the aerosol chamber at the level of the vibrating meshes so as to avoid any uncontrolled interferences. One skilled in the art can optimize the shape and the number of dividing walls 122 for this purpose.

According to a specific embodiment, such a dividing wall 122 can also comprise a heating element acting as an aerosol heater 130. In case such an aerosol heater 130 is present, it can be thus be arranged between the vibrating meshes 14 so as to limit or avoid the interferences and the corresponding uncontrolled effects on the flowrate of the solution S. Such heating element 130 can be designed as a coil oriented vertically inside the aerosol chamber 13 at the level of the vibrating meshes 14.

According to an embodiment, the frequency of the vibrating meshes 14 can be controlled, either individually or globally. For example, all the vibrating meshes 14 can vibrate at a same given frequency. Some adjacent vibrating meshes 14 can vibrate out of phase or with a predetermined phase difference or with opposite phase. By this way, the uncontrolled effects of the phase interferences on the flow rate of the solution are avoided. In case the flow rate is changed by the user, by changing the frequency of vibration, all the vibrating meshes 14 maintain their phase difference.

According to another embodiment, better represented by FIG. 5a, the preheating chamber 12 can extend along the aerosol chamber 13 on a distance longueur than the storage compartment 11. For example, the preheating chamber 12 can occupy a portion of the lateral wall 121 larger than the portion of the lateral wall 121 occupied by the storage compartment 11 by a factor of 30% or 50% or 100% or even 200% or more. Several vibrating meshes 14 can then be arranged along the length of the aerosol chamber 13. This configuration allows to improve the flow of particles 5 toward the aerosol chamber 13. Such a configuration does not excludes that several vibrating meshes 14 can be provided at a given height of the aerosol chamber 13, as above described.

Depending on the configuration and the dimensions of the preheating chamber 12 and the aerosol chamber 13, as well as the places of the vibrating meshes 14, the phase difference between adjacent vibrating meshes 14 can also be object of a specific control. For example, instead of, or in addition to, modifying the frequency of vibration, the user may modify the phase difference between adjacent meshes to adjust the flowrate. For example, a completely opposite phase between two adjacent vibrating meshes 14 can be selected to provide a maximal flowrate. Decreasing the phase difference can allow to decrease the flow rate. In case where facing vibrating meshes 14 have no phase difference, they can compensate each other and provide no significant flowrate.

For a better control, the phase difference can be modified according to the selected frequencies, meaning that the phase difference is adjusted according to the frequency or to a range of frequencies of vibration of the vibrating meshes 14.

Only one of the dividing wall 122 and the frequency control or phase difference control of the vibrating meshes 14 can be considered. Alternatively, a combination of dividing wall 122 and a control of one or both of the frequency and the phase difference can be considered according to the needs.

Alternatively or in addition to the frequency control, the amplitude of vibration of the vibrating meshes 14 can also be object of a control, either individually or globally.

According to another embodiment, better shown in FIG. 5b, the device 1 comprises one or more piezo pump 125 in addition or in replacement of one or more vibrating mesh 14. Such a piezo pump 125 is arranged in the preheating chamber 12. It can be combined to a vibrating mesh 14 for a better volatilization efficiency. It can further be combined to a heating device 120 so as to form an assembly comprising a piezo pump, a heating device 120 and a vibrating mesh 14.

The end part of the aerosol chamber 13 can be provided with a means to collect the condensed fluids 15. Such a means may be limited to a porous portion or a capillary assembly at the end of the aerosol chamber 13. Alternatively or in addition, one or several vibrating meshes can be provided to collect the residues and project them back to the aerosol chamber 13. Such vibrating meshes can have predetermined frequency and amplitude, which are preferably constant and not modulable. This does not excludes that one or both of the frequency and amplitude can be modified for some specific needs, for example during a washing process or a maintenance process.

The air inlet 6, better represented in FIG. 5b, although it may be present in the other embodiments represented by FIG. 4a, 4b or 5a, is preferably arranged at a level of the aerosol chamber 13 comprising the volatilizing elements, meaning at a bottom part of the aerosol chamber 13.

The overall design of the present device can allow to remove one or several of its elements. It can be for example modular, having a first removable part comprising the storage compartment, a second removable part related to the pre-heating chamber 12, a third removable part corresponding to the volatilizing element, and a fourth part related to the aerosol chamber 13. By this way, any of these modular parts can be independently separated from the others and clean up and/or replaced. According to a specific embodiment, the volatilizing element, or at least the meshes 14 it comprises, can be easily dissociated from the device 1 by the user so as to be cleaned up or replaced by a new one. This facilitates the maintenance of the device 1 by the user and allows him to maintain the proper volatilization conditions of the solution S, without degradation of the taste due to potential accumulation of residues. It is however highlighted that due to the absence of burn, the device 1 normally does not contain residues of liquid degradation or burn.

Alternatively or in addition, the device 1 can allow one or several modes related to either the disinfection or the cleanup of at least some of the parts of the device 1 using a dedicated cleaning solution. Such a cleaning solution could be for example an acidic solution, or a basic solution, an antiseptic solution, an alcoholic solution or a selection of such solutions to be used in a precise order. In a cleaning mode, the cleaning solution can be included to the storage compartment 11 for a circulation in the complete circuit of the device.

The device according to the present disclosure further comprises a command input 22 such as an input button or a combination of input button, or a tactile display, or any related input command that a user can activate. The command input is connected to a command unit 20 adapted to modulate the heating device 120 of the pre-heating chamber 12. The command unit 20 may in addition allow the modulation of the frequencies of the vibrations of the mesh 14 of the volatilizing element. The command unit 20 may in addition allow the modulation of the aerosol heater 130 or the plurality of aerosol heaters. The command unit may in addition allow to modulate the fresh air intake by closing more or less the air inlet 6. The device 1 can in addition comprise a display 21 allowing the user to input data and/or receive information on the status of the device 1. The device 1 may in addition comprise a communication unit 30 allowing to share some data with a remote system. Such a remote system may be for example a platform providing services or information to the user of the device, such as notice of use, or specific settings with regard to the liquid compositions S. The remote system may allow to automatically update the command unit 20. Such updates can for example relate to better processes involved in the mutual management of the heating device 120 and the frequency of vibrations of the volatilizing element. The remote system may alternatively designate a mobile phone or another terminal of the user, such as his computer.

The input command 22 can allow the user to choose a debit or a predetermined quantity of aerosol 5 or any other parameters. For ease of use, the command unit 20 may automatically select the proper combination of temperatures of the heating device 120, aerosol heater 130 and frequency of vibration of the volatilizing element to provide the user the requested results. In addition, the command unit 20 may automatically select the proper combination of temperatures of the heating device 120 and frequency of vibration of the volatilizing element so that a minimum energy is consumed. For example, heating of the heating device 120 lower than necessary to provide the suitable viscosity of the liquid composition S and compensated by an increase of the vibration frequency of the volatilizing element if the global energy consumption appears lower for a given result. The activation of the heating device 120 and the volatilizing element may also be adapted according to the autonomy of the device 1. It is here highlighted that the device 1 can comprise a battery and a plugging means to recharge the battery.

The input command 22 may in addition allow to select a cleanup mode or a maintenance mode or any other specific mode of the device 1.

The device according to the present disclosure thus allows to provide aerosol having thin microdroplets based on liquid compositions S having a relatively high viscosity. It furthermore allows such a microdroplet generation with a minimum of energy consumption thanks to the combination of the heating element 120 and the vibrating mesh 14. Liquid compositions having high viscosity necessitate stronger vibrations to produce microdroplets, thus consuming more energy. Even under such conditions, the size of the produced microdroplets may not be so homogenous or so thin compared to the present aerosol, which comprises a pre-heating chamber 12 upstream the volatilization element.

The present invention further relates to a kit comprising a device 1 and at least one liquid composition S. The liquid composition S may be separately packed in a tank adapted to fill or refill the storage compartment 11. The liquid composition S may be separately packed in a cartridge adapted to be connected to the device 1. Such a cartridge may comprise a readable device such as a RFID or any similar device allowing to automatically identify the nature of the liquid composition, including its viscosity at a given temperature, and/or its expiration date. Other characteristics may be stored in the readable device. The readable device may be automatically read by the device 1 so that the proper settings are suggested to the user through the display 21 or automatically applied. The kit can comprise additional liquid compositions S, offering a large variety of choice to the user. In case the liquid composition comprises a medical treatment or any pharmaceutical compounds, the kit can comprise in addition the corresponding notice of use.

The liquid compositions S of the present invention are preferably aqueous compositions. The liquid composition S thus comprise more that around 60% by weight of water, preferably more that 70% by weight or even more preferably more than 80% by weight of water, this amount being determined with regard to the total weight of the liquid compositions S. The water may be mineral water, distilled water, pure water, or water having pharmaceutical quality such as water for injection. To this end, the complete liquid compositions S may be subjected to sterilization processes.

The liquid compositions S of the present invention further comprises one or more substances having biological effect through inhalation. Such substances include for example Ventolin or any other drugs commonly used to treat respiratory symptoms or diseases like asthma, as well as acceptable salts thereof. The substance of the liquid compositions S may also be or include nicotine, also known as Pyridine-3-(1-methyl-2-pyrrolidinyl). The substance may designate nicotine derivatives or salts thereof. Nicotine derivatives include for example 4-nitronicotine-N-oxide, 4-aminonicotine, nornicotine, anabasine, myosmine, nicotirine, and anatabine. The nicotine or nicotine derivatives may be combined to a salt selected from the group of sulfate, tartrate, Hexafluorosilicate, hydrochloride, diformate, triacetate, tri propionate, tri butyrate, isovalerate, di nitrophenolate, dipicrate, dihydrochloride, hydroiodide, picrolonate, phthalate, dipicrolonate, aurichloride, benzoic, levunilic, pyruvic or a combination thereof.

The substance may alternatively be or comprise a natural oil or a natural plant extract. For example, the substance may be a cannabinoid such as Tetrahydrocannabinol (THC), Cannabidiol (CBD), Cannabinol (CBN), Cannabigerol (CBG), Tetrahydrocannabivarin (THCV), cannabidivarin (CBDV), Cannabichromene (CBC).

The substance of the liquid compositions S may be comprised between 1% by weight and 10% by weight of composition.

The liquid compositions S may further comprise a polyol, in particular a diol. Such polyol may be selected in the group of propylene glycol (propane-1,2-diol), vegetol (Propane-1 3 diol), glycerine (propane-1,2,3-triol), Propane 1-1-diol, Propane 2-2 diol, Butane 1,4 diol, Butane 1,3 diol, Butane 2,3 diol and Pentane 1,5 diol or a mixture thereof. The amount of the polyol is comprised between 3 and 40%, preferably between 5 and 35% by weight of liquid composition S.

To transfer the flavor from water solution, a flavor enhancer may be required. Such polyol may be used as flavor enhancer. Preferred polyols used as flavour enhancer can be selected among the propylene glycol (PG), the vegetal glycerin (VG). For example, a combination of propylene glycol (PG) and vegetal glycerin (VG) can be used in a ratio of PG/VG of around 80/20, around 70/30, around 60/40, around 50/50 can be used. Some solution having more VG than PG can also be used, such a ratio PG/VG of around 40/60, around 30/70 or around 40/60. The ratios can be determined by volume. The ratios can also be determined by weight.

The amount of polyol in the solution S can impact its flowrate in the device 1. According to a preferred embodiment, the ratio of polyol/water is comprised between 10/90 by volume and 20/80 by volume. Such solutions are preferably stored in a sterile closed tank. Such a sterile closed tank can be directly engaged within the device 1 in a way to feed the preheating chamber 12 and produce the particles 5.

Solutions comprising a ratio polyol/water of 20/80 by volume or more, such as 30/70 or 40/60 by volume, can however still be used in the present device 1. In particular, despite a possible increase of the viscosity of such solutions due to the increased proportion of polyol, the adequate flowrate can be obtained thanks to the above-described arrangement. Solutions comprising a ratio of polyol/water of 20/80 or more have in addition the advantage to be less sensitive or not sensitive to bacteria development. Such solutions do not need to be sterilized and encapsulated in a sterile closed tank. Such solutions can then be stored in recipients which can be used to refill the storage compartment 11 of the device according to the needs without the necessity to systematically engage a closed capsule. This being said, the presently claimed device is particularly well adapted to receive a solution wherein the ratio polyol/water is either lower than 20/80 by volume or equal or above 20/80 by volume.

For e-cigarette, aqueous solutions may not transmit taste of nicotine and aroma in a proper manner. To improve the transmission of the taste to the user, a combination of physical particle size reduction and a flavor enhancer, which is also used as surfactant, can be used. The combination of particle size reduction and flavor enhancer may thus be required to properly transmit the taste.

The liquid composition S of the present invention may comprise one or several flavors. It may in addition comprise preservative compounds such as bactericides (triacetine), fungicides, and anti-oxidants.

The present invention further comprise a method of vaporizing an liquid solution into an aerosol adapted for inhalation, the liquid composition S having a viscosity higher than 2 or 3 or 5 Cp at 20° C. The present method comprises a heating step a) of continuously and instantaneously pre-heating a liquid solution S at a temperature lower that its boiling point, so that no vapor is produced and no product degradation occurs. The pre-heated solution S is directly submitted to a spreading step b) of volatilizing the pre-heated liquid solution S into microdroplets 5 having an average diameter of less than 4 μm, like around 1 μm.

The steps a) and b) preferably occurs concomitantly. They may be manually initiated by the user by means of a command button 22.

The method of the present invention further comprises a modulation step c) of modulating one or more of the temperature of the pre-heating chamber 12, the frequency of vibration of the volatilizing element, and the temperature of the aerosol heater. The modulation step c) includes a debit selection by the user and the automatic modulation of the heating device 120 and the frequency of vibration of the volatilizing element by the command unit 20. In case several vibrating actuators 14 are present, the modulation step c) may allow to sequentially activate the vibrating actuators to increase or reduce the flowrate of the liquid composition toward the aerosol. One or several vibrating actuators can be independently activated.

### REFERENCE NUMBERS AND SYMBOLS

1 Device
10 Housing
11 Storage compartment
110 Solution inlet
12 Preheating chamber
120 Heating device
121 Lateral wall
122 Dividing wall
125 Piezo pump
13 Aerosol chamber
130 Aerosol heater
14 Vibrating mesh
15 Means for collecting residues
20 Command Unit
21 Display
22 Command Button
30 Communication unit
5 Droplets
6 Air inlet
8 Heated aerosol
9 Mouth port

1. Device comprising a housing having a storage compartment adapted for the storage of a liquid compositions, a volatilizing element adapted to volatilize the liquid composition into microdroplets, and an aerosol chamber,
characterized in that: wherein the device further comprises at least one pre-heating chamber downstream said storage compartment and in fluid communication with said storage compartment by means of at least one solution inlet, said at least one pre-heating chamber comprising one or more heating device and being arranged upstream said volatilizing element, in that said aerosol chamber is downstream the volatilizing element, said aerosol chamber comprising, or being connected to, a mouth port, wherein the preheating chamber and the aerosol chamber are arranged in the continuity from one another having said volatilizing element along a transversal position therebetween, or wherein the preheating chamber takes the form of one or several lateral spaces surrounding the aerosol chamber having said volatizing element arranged on the corresponding lateral walls, so that said microdroplets have a mean diameter equal or lower than 4 micrometers.

2. Device according to claim 1, said heating device being adapted to heat the liquid composition at a temperature lower than its boiling point so that it provides no increase, or substantially no increase of pressure.

3. Device according to one of the preceding claim 1, said volatilizing element comprising at least one vibrating mesh comprising one or several flat porous membranes having pore size of around 1 μm or lower, combined with at least one piezoelectric or magnetoelectric actuator, or comprising at least a piezo pump or a combination of both.

4. Device according to claim 3, wherein said volatilizing element denotes or comprises one or more vibrating mesh adapted to vibrate at frequencies comprised between around 50 KHz and around 150 KHz or between around 500 KHz and around 1500 KHz.

5. Device according to claim 1, wherein said volatilizing element denotes or comprises one or more vibrating mesh comprising a non-flat porous material adapted to present an increase contact surface with said liquid composition (S) and/or a multilayer porous material.

6. Device according to claim 1, wherein said pre-heating chamber comprises one or several capillaries or a porous material driving the liquid composition in contact to said volatilizing element.

7. Device according to claim 1, said aerosol chamber, further comprising, or being connected to, an air inlet adapted to combine the microdroplets with external air under inhalation via the mouth port.

8. Device according to claim 1, wherein the aerosol chamber further comprises at least one aerosol heater.

9. Device according to claim 1, wherein said at least one preheating chamber takes the form of one or several lateral spaces surrounding said aerosol chamber, wherein said volatizing element comprises one or several vibrating meshes arranged on the corresponding lateral walls.

10. Device according to claim 9, comprising more than one vibrating meshes facing each other, and further comprising at least one internal wall positioned between said vibrating meshes.

11. Device according to claim 10, wherein said internal wall denotes a heating element acting as an aerosol heater.

12. Device according to claim 10 comprising several preheating chambers surrounding said aerosol chamber corresponds to the interstitial space between said preheating chambers or between said preheating chambers and the housing or both.

13. Device according to claim 1, further comprising a means for collecting residues, such means comprising one or several of a porous material, a capillary assembly and at least one vibrating mesh.

14. Device according to claim 1, further comprising a command input adapted for modulating the temperature of the pre-heating chamber, or one or both of the frequency and amplitude of vibration of the volatilizing element when denoting one or more vibrating meshes or the combination of the temperature of the pre-heating chamber and one or both of the frequency and amplitude of vibration of the volatilizing element, when denoting one or more vibrating meshes.

15. Device according to claim 1, said volatilizing element comprising more than one vibrating mesh, wherein at least some of adjacent vibrating meshes vibrate with a controlled phase difference.

16. Device according to claim 15, wherein said phase difference can be adjusted with respect to the frequency of vibration or a range of frequencies of vibration.

17. Method of volatilizing a liquid composition into microdroplets having a diameter lower than 4 micrometers, by means of a device according to claim 1, the liquid composition having a viscosity higher than 2 cP at 20° C., the method comprising a heating step a) of pre-heating said liquid composition at a temperature lower that its boiling point, so that no vapor is produced, and a spreading step b) of volatilizing the pre-heated liquid composition into microdroplets having an average diameter lower than around 4 μm.

18. Method according to claim 17, further comprising the modulation step c) of modulating the temperature of the pre-heating chamber and/or one or more of the frequency, amplitude and phase difference of vibration of the vibrating mesh and/or the number of vibrating actuators when applicable.

19. Method according to claim 17, wherein said liquid composition comprises more than 60% of water and having viscosity higher than 2 cP at 20° C., between 1 and 10% of a substance having a biological effect under inhaling conditions, and between 5 to 40% of a polyol, said liquid composition being substantially free of additional organic solvent.

20. Method according to claim 19, wherein the ratio polyol/water in said liquid composition is comprised between 10/90 and 20/80, and wherein said composition is stored in a sterile closed tank adapted to be engaged within the device.

21. Method according to claim 19, wherein the ratio polyol/water in said liquid composition is more than 20/80 and wherein said liquid composition is stored in a non-sterilized recipient usable to refill the device.

22. Method according to claim 19, said liquid composition further comprising one or several flavours.

23. Method according to claim 17, said substance being selected from nicotine, a nicotine derivative, Tetrahydrocannabinol (THC) or cannabinoid family or a pharmaceutically acceptable salt thereof.
